(19) United States
(12) Patent Application Publication
(10) Pub. No.: US 2026/0171258 A1
(43) Pub. Date: Jun. 18, 2026

(54) PATIENT ANXIETY AND VITAL SIGNS MONITORING WITH ARTIFICIAL INTELLIGENCE

(71) Applicant: A-DEC, INC., Newberg, OR (US)

(72) Inventor: Ryan Michael WILLIAMS, Sherwood, OR (US)

(21) Appl. No.: 19/415,571

(22) Filed: Dec. 10, 2025

Related U.S. Application Data
(60) Provisional application No. 63/735,026, filed on Dec. 17, 2024.

(51) Int. Cl. G16H 50/30; G06V 40/16; G06V 40/18; G10L 25/66; G16H 20/70; G16H 40/67

(52) U.S. Cl. CPC G16H 50/30; G06V 40/174; G06V 40/18; G10L 25/66; G16H 20/70; G16H 40/67

(57) ABSTRACT

Embodiments herein relate to identifying a status of a patient such as in a dental or other medical treatment room. In one aspect, the solutions provide a system to determine whether the patient is anxious and/or a degree of anxiety of the patient. The solutions can include training a model such as a large language model (LLM). In a training phase, data is gathered from patients, the environments of treatment rooms and equipment of the treatment rooms during various medical procedures for a patient population. The model is then trained to correlate the data with a degree of anxiety. Once the model is trained, it can be deployed to determine whether an individual patient is anxious and to take appropriate countermeasures to reduce anxiety.

### CROSS-REFERENCE TO RELATED APPLICATIONS

This application claims the benefit of priority to U.S. Provisional Patent Application No. 63/735,026, entitled “Patient Anxiety And Vital Signs Monitoring With Artificial Intelligence,” filed Dec. 17, 2024, which is incorporated herein by reference.

### TECHNICAL FIELD

Embodiments herein relate to identifying patient anxiety such as in a dental or other medical treatment room.

### BACKGROUND

Many people avoid visiting a dentist or other medical professional, or experience stress during such visits, due to anxiety. This can result in negative health consequences. The medical professional can attempt to reduce anxiety with approaches such as providing soothing music or decorating the office with plants or soothing colors. The medical professional can also offer sedatives such as nitrous oxide. However, addressing patient anxiety is an ongoing challenge in healthcare delivery.

### BRIEF DESCRIPTION OF THE DRAWINGS

Embodiments will be readily understood by the following detailed description in conjunction with the accompanying drawings and the appended claims. Embodiments are illustrated by way of example and not by way of limitation in the figures of the accompanying drawings.

FIG. 1 depicts a system 100 for determining the status of a patient, in accordance with various embodiments.

FIG. 2 depicts an example of the dental operatory/treatment room equipment 130 of FIG. 1, in accordance with various embodiments.

FIG. 3 depicts an example user interface 300 for use in the system of FIG. 1, in accordance with various embodiments.

FIG. 4 depicts an example dental tool 400 with a user interface 410 for use in the system of FIG. 1, in accordance with various embodiments.

FIG. 5 depicts an example implementation of the system of FIG. 1, in accordance with various embodiments.

FIG. 6 depicts an example computing device for use in the system of FIG. 1, in accordance with various embodiments.

FIG. 7 depicts an example process to train a model to correlate the status of a population of patients to health data, equipment data and environmental data, in accordance with various embodiments.

FIG. 8 depicts an example process to use a model to determine the status of an individual patient based on health data, equipment data and environmental data, in accordance with various embodiments.

### DETAILED DESCRIPTION OF DISCLOSED EMBODIMENTS

In the following detailed description, reference is made to the accompanying drawings which form a part hereof, and in which are shown by way of illustration embodiments that may be practiced. It is to be understood that other embodiments may be utilized and structural or logical changes may be made without departing from the scope. Therefore, the following detailed description is not to be taken in a limiting sense, and the scope of embodiments is defined by the appended claims and their equivalents.

Various operations may be described as multiple discrete operations in turn, in a manner that may be helpful in understanding embodiments; however, the order of description should not be construed to imply that these operations are order dependent.

The description may use perspective-based descriptions such as up/down, back/front, and top/bottom. Such descriptions are merely used to facilitate the discussion and are not intended to restrict the application of disclosed embodiments.

The terms “coupled” and “connected,” along with their derivatives, may be used. It should be understood that these terms are not intended as synonyms for each other. Rather, in particular embodiments, “connected” may be used to indicate that two or more elements are in direct physical contact with each other. “Coupled” may mean that two or more elements are in direct physical contact. However, “coupled” may also mean that two or more elements are not in direct contact with each other, but yet still cooperate or interact with each other.

For the purposes of the description, a phrase in the form “A/B” or in the form “A and/or B” means (A), (B), or (A and B). For the purposes of the description, a phrase in the form “at least one of A, B, and C” means (A), (B), (C), (A and B), (A and C), (B and C), or (A, B and C). For the purposes of the description, a phrase in the form “(A)B” means (B) or (AB) that is, A is an optional element.

The description may use the terms “embodiment” or “embodiments,” which may each refer to one or more of the same or different embodiments. Furthermore, the terms “comprising,” “including,” “having,” and the like, as used with respect to embodiments, are synonymous, and are generally intended as “open” terms (e.g., the term “including” should be interpreted as “including but not limited to,” the term “having” should be interpreted as “having at least,” the term “includes” should be interpreted as “includes but is not limited to,” etc.).

With respect to the use of any plural and/or singular terms herein, those having skill in the art can translate from the plural to the singular and/or from the singular to the plural as is appropriate to the context and/or application. The various singular/plural permutations may be expressly set forth herein for sake of clarity.

As mentioned at the outset, various challenges are presented in addressing patient anxiety in a dental operatory/treatment room despite existing countermeasures.

The solutions provided herein address the above and other issues.

In one aspect, the solutions provide a system to determine the status of a patient such as to determine whether the patient is anxious and/or to determine a degree of anxiety of the patient. The solutions can include training a model (e.g., one or more models) such as including a large language model (LLM), a type of artificial intelligence (AI). In a training phase, data is gathered from patient sensors, equipment, and the environment during various medical procedures for a patient population. The model is then trained to correlate the data with a degree of anxiety.

Once the model is trained, it can be deployed for use in determining whether an individual patient is anxious. The model can continue to be trained as new data becomes available. Data is gathered from the individual patient during their office visit, e.g., before and/or during a procedure, or even after as well, and a determination is made of their anxiety level. The level can be coded in various ways such as by a number or a color and presented on a user interface (UI). The UI can be a screen such as a phone or laptop screen, and/or integrated into the medical equipment such as the console or a hand-held tool. The UI can be provided for the patient and/or one or more medical providers. Patients can respond to an indication of their anxiety by performing actions such as intentionally calming themselves with breathing exercises, focusing on a distraction such as music or a video screen, or communicating with the dentist.

Once it is determined that the patient is anxious, one or more anxiety-reducing countermeasures can be performed. The countermeasures can be started automatically by one or more computing devices, without user (patient or provider) interaction. In other words, the countermeasures can be generated automatically or through patient and/or provider interaction. Such countermeasures include modifying the temperature of the room, adjusting the temperature of the chair or environment around the chair, providing soothing sounds (e.g., music or white noise), images, or scents, adjusting the dental operatory light or the environmental (overhead) lights, or driving patient entertainment such as audio/video on a screen or a virtual reality headset.

The solutions provide a number of advantages, including reducing patient anxiety as well as stress experienced by the provider, who may be affected by the patient's anxiety. Patient treatments can be expedited when patients are calm, and the need for follow-up visits may be reduced.

The above and other features can be understood further in view of the following discussion.

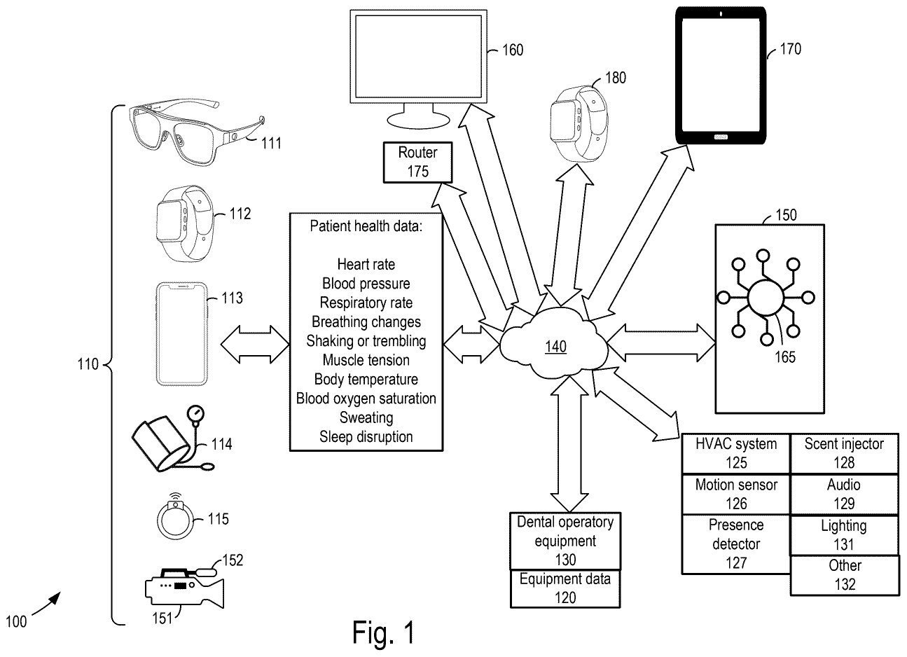

FIG. 1 depicts a system 100 for determining the status of a patient, in accordance with various embodiments. The system is for a scenario involving a dental office, as an example. A patient may visit a dental office for procedures such as cleanings, fillings, extractions, crowns, root canals, and oral exams. The system could be used in other medical scenarios as well, such as when a patient visits a provider for a checkup or a procedure. The system includes patient health monitoring devices 110 which may be, e.g., worn or carried by the patients, such as a fitness tracker (also referred to as a health tracker or smart wearable), smart glasses, and a smart phone 113. Some fitness trackers can obtain health data or vital signs such as heart rate, respiratory rate, body temperature, blood oxygen level, sleep duration and quality, and electrical activity of the heart such as an electrocardiogram (ECG). Fitness trackers can have different formats. For example, they can be wrist-worn in the format of a watch 112, or finger-worn in the format of a ring 115. Smart glasses 111 may have similar capabilities. The fitness tracker and smart glasses may operate in connection with a smart phone or other UI to display data and connect via a network to a server.

In one approach, the patient health monitoring device can enter a mode that provides specific health data based on the medical office that the patient is at, and/or based on the procedure that the patient is undergoing or will undergo. For instance, the device 110 may have a dental mode for when the patient is in a dental office. The device 110 can enter a mode based on a command from the user, or automatically, e.g., by recognizing the location. For example, the location can be recognized by a global positioning system (GPS) of the device, or by an identifier of a Wi-Fi signal from a router in the medical office. A calendar function of the device could also indicate the patient is at a specific location at a specific day/time and trigger a corresponding mode. The location could also be recognized by the wearable device by detecting a radio beacon in the office such as from a Bluetooth Low Energy (BLE) beacon.

The device 110 could be a personal item that the user wears in their daily lives or it could be a device provided by the medical office, e.g., to monitor the patient or for other use during a procedure, whether worn by the patient or otherwise positioned on or near the patient.

Another example of the patient health monitoring device 110 is a blood pressure cuff 114.

The patient health monitoring devices are examples of patient sensors. The camera 151 and a microphone 152 are also patient health monitoring devices that obtain images and sounds from the patients, for use in determining the patient's status. The microphone can also be used to obtain sounds from powered tools used in the treatment room, to identify a tool being used. The camera can also potentially be used to obtain images of powered tools used in the treatment room, to identify a tool being used.

Equipment data 120 can also be obtained from the dental operatory equipment and used to determine a status of the patient. This data can be obtained from sensors and settings that are associated with the medical equipment 130 such as in a dental operatory. The equipment data can indicate a status, setting, or configuration of equipment in the treatment room. An example of an equipment sensor is a pressure sensor in a chair used to detect the patient's movement in the chair, which can be an indicator of their status. An example of an equipment setting is a setting of a dental operatory light such as its brightness and color. For example, a light may have a switch which can be set to different brightness settings so that the current setting is known by a control circuit within or associated with the light. Alternatively, a sensor such as a light sensor could be used to determine the brightness and color of the light. Other examples of equipment settings include the angle of recline of the patient chair, a type of tool being used and a speed of the tool.

The data from the patient health monitoring devices and the equipment together can provide patient health data such as heart rate, blood pressure, respiratory rate, breathing changes, shaking or trembling, muscle tension, body temperature, blood oxygen level (oxygen saturation level), presence or amount of sweating, and data regarding sleep disruption. The health data can be provided as snapshot values and/or as a trend such as an increasing or decreasing trend.

For example, when a person is stressed or anxious, their heart rate, blood pressure, respiratory rate, breathing changes, shaking or trembling, muscle tension, body temperature, and/or presence or amount of sweating can increase. Blood oxygen level can decrease due to a stress response causing faster, shallower breathing that reduces the amount of oxygen delivered to the bloodstream.

A patient health monitoring device can detect heart rate using photoplethysmography (PPG), for instance, which relies on optical sensors. The sensors use light-emitting diodes (LEDs) to shine light through the skin and measure the amount of light that is reflected back by a photodetector. The volume of blood in the vessels changes with each heartbeat, changing the light absorption and reflection, and thereby allowing the device to calculate the heart rate.

A patient health monitoring device can detect blood pressure in different ways. One approach uses an accelerometer to detect a pulse wave's arrival at the wrist, for example. An optical sensor is used to measure the time it takes to travel from the heart to the wrist as a Pulse Transit Time (PTT), which is inversely related to blood pressure.

Another approach uses light to measure changes in blood volume in superficial arteries. By analyzing the shape of a PPG waveform, which is influenced by blood pressure, an estimate of blood pressure can be obtained.

Another approach uses seismocardiography to detect tiny chest wall vibrations created by heartbeats using an accelerometer, such as when the wearable device is held against the chest.

Generally, various sensor signals can be analyzed by machine learning algorithms at the wearable device to identify features that correlate with blood pressure.

A patient health monitoring device can estimate respiratory rate from patterns of PPG signals or from heart rate variability, for instance.

A patient health monitoring device can detect breathing changes by analyzing variations in a PPG signal, which measures blood volume, and by using accelerometers to sense subtle body movements. The changes in intrathoracic pressure from each breath alter blood flow to the heart, which creates baseline fluctuations in the PPG signal that the device can track. Accelerometers can detect the physical motion of breathing, particularly when combined with advanced signal processing and machine learning algorithms to isolate these specific movements from other body motions.

A patient health monitoring device can detect shaking or trembling using accelerometers and gyroscopes that measure movement, and/or via electromyography (EMG) sensors that detect electrical activity in muscles.

A patient health monitoring device can detect muscle tension using electromyography (EMG), which measures the electrical activity produced when muscles contract, or forcemyography (FMG), which senses changes in muscle stiffness and volume. Piezoelectric pressure sensors can also be used to measure the physical indentation or surface resistance changes caused by muscle movement.

A patient health monitoring device can detect body temperature using skin-temperature sensors that measure infrared radiation and thermistors that measure resistance changes.

A patient health monitoring device can detect the presence or amount of sweating using electrochemical sensors that measure the electrical signals generated when sweat components such as electrolytes (e.g., Na⁺ or K⁺) and metabolites (e.g., glucose or lactate) interact with the sensor. They can also use conductivity changes to estimate the amount of sweat or employ optical methods to detect the formation of a sweat film on the skin.

A patient health monitoring device can detect blood oxygen level using LEDs that shine light through the blood in the wrist, for example. By measuring how much light is reflected back to a sensor and using algorithms to analyze this data, the device can estimate the color of the blood and calculate the blood oxygen saturation level. Different wavelengths of light, including red and infrared, are used because oxygenated and deoxygenated blood absorb and reflect light differently. Another approach uses an oximeter attach to the finger, earlobe, or other body part, for example.

The camera 151 is also provided in this example to provide images which can be used by the model 165 to monitor the patient in terms of, e.g., eye movement, pupil dilation, blinking, and/or periods of closing the eyes. A relatively large amount of, e.g., eye movement, pupil dilation, blinking, and/or periods of closing the eyes may correlate with an anxious state. The camera provides video or still image data of the patient's eyes, which can be transmitted to the model 165 and server 150 for processing, in one approach. The server 150 can be at the treatment site or remote from the treatment site. The server 150 is an example of one or more computing devices.

In one approach, pupil-corneal reflection (P-CR) is used. In this approach, a light associated with the camera shines a near-infrared light on the eye, and the camera captures images of the reflections from the cornea and the pupil. Algorithms can be used to assess these images to track the pupil's position, its size, and the reflections to determine where the person is looking, detect blinks when the pupil disappears, and gauge pupil dilation. The camera images can be used to detect eye blinking and/or periods of closing the eyes by analyzing the ratio of eye-opening measurements, such as the distance between the eyelids, and comparing it to the eye's open state.

The camera 151 could also obtain images of the patient's face to recognize their status based on facial expressions. In one approach, the patient's facial expression is compared to a database of expressions at the model 165 that are correlated with different anxiety levels, to determine the current anxiety level.

Generally, the model 165 can correlate a person's facial expression with their level of anxiety using a combination of computer vision, machine learning, and established psychological frameworks. The process involves capturing video, identifying specific facial movements, extracting relevant data, and using trained AI models to classify or quantify the anxiety level.

The camera first detects the face in its field of view. Computer vision algorithms (e.g., the Viola-Jones algorithm or deep learning models) then identify and track key facial landmarks, such as eye corners, eyebrow positions, mouth shape, and nose tip.

The system analyzes the movement and positioning of these landmarks over time. Instead of just analyzing static images, the focus is on dynamic changes and micro-expressions. Features extracted can include:
Eye activity: Blink rate, eyelid lifting, pupil dilation, and gaze spatial distribution.
The eyes may widen when a person is fearful or anxious.
Mouth activity: Frequency of mouth opening and closing, and specific mouth shapes related to emotions. The mouth may be open when a person is fearful or anxious.
Eyebrow movements: Inner brow raising or lowering.
Head movements: Nodding, bobbing, or constrained movements.

The extracted movements can be mapped to the Facial Action Coding System (F.A.C.S.), a standardized system that breaks down facial expressions into specific “action units” (e.g., AU1: inner brow raiser). Specific AUs have been identified as indicators of anxiety and stress, such as elements of fear expressions, increased eye blinks, and higher frequency of lower lip movement.

The collected data (e.g., features and AUs) can be fed into a machine learning model (e.g., Convolutional Neural Networks or Support Vector Machines, as represented by the model 165) that has been trained on large, annotated datasets of individuals exhibiting various levels of anxiety. These levels of anxiety can be correlated with self-report scales such as the Hamilton Anxiety Scale or Depression, Anxiety, and Stress Scale (DASS)-21.

The model identifies patterns in the facial cues that correlate with anxiety. The output is a classification of an emotional state (e.g., “anxious,” “stressed,” “neutral”) or a continuous score indicating the intensity or severity of anxiety.

Additionally, specific facial cues can be associated with anxiety, including more facial movements related to fear and arousal, an increase in total facial movement during high anxiety states, a higher proportion of repetitive lower lip movement, an increased frequency of eye blinks and/or specific eyebrow movements, and constrained head movements in some cases.

In another option, the camera 151 could also obtain images of the patient's body position to recognize their status. The patient's body position can be compared to a database of body positions at the model 165 that are correlated with different anxiety levels, to determine the current anxiety level. For example, a higher anxiety level can be associated with postures such as slouching or hunching, a closed-off position such as crossing the arms or legs, looking down, and fidgeting or restlessness including shifting, swiveling, or tapping the feet or hands. A lower anxiety level can be associated with postures such as upright and open such as sitting tall with shoulders back, and non-crossed arms or legs, and maintaining a stable seated position without excessive movement.

The microphone 152 can also be provided at a location near the patient to monitor the patient's voice. Audio detected from the microphone can be analyzed at an audio analysis system at the model 165 and the server 150, for example, to detect and process the patient's voice at various times when the patient is speaking with the dentist or other medical professional, for example. Anxiety can be detected from a person's voice through changes in speech patterns and vocal quality. These include speaking faster or softer, mumbling, a higher pitch, and a breathy or strained tone due to muscle tension in the throat. AI techniques can be used to identify these vocal differences. Specifically, vocal changes associated with anxiety include: pace and volume, e.g., speaking more quickly, softly, or barely audibly, pitch and tone, e.g., a higher-pitched, thinner, or more breathy voice, clarity, e.g., mumbling or a lack of vocal power and control, rhythm, e.g., rushing through sentences or words, and vocal quality, e.g., tightness or a strained feeling in the voice due to muscle tension.

In further detail, increased muscle tension in and around the voice box (larynx) can cause the vocal folds to constrict, resulting in a higher-pitched or inconsistent voice. Adrenaline and general muscle tension can cause the voice to sound shaky or trembled. A person might speak more rapidly, or conversely, have difficulty forming words, leading to stuttering or hesitations. Shallow or poor breath control due to anxiety can lead to a breathless quality, a strained voice, or a feeling of a “lump in the throat”. Increased pauses, the use of more filler words (“umm,” “uhh,” “like”), and difficulty organizing thoughts clearly can disrupt the flow and fluency of speech. The voice may become quieter due to a lack of breath support, or in some cases, louder due to increased muscle tension.

A change in a patient's voice, such as speaking more rapidly, can be detected relative to an average value (e.g., speech rate such as words per minute) among a general population or can be detected relative to a previous value which is specific to the patient, for example, as recorded in one or more previous visits. In a dental operatory, the patient's voice may be captured while the patient is in the chair or at another location in the treatment room, in connection with a dialogue with the provider.

Machine learning algorithms can analyze these acoustic and linguistic features. Examples of AI voice analysis tools are available from Kintsugi Health, Berkely, California, and Ellipsis Health, San Francisco, California.

A heating, ventilation and air conditioning (HVAC) system 125 of the treatment room of the medical provider may also be in communication with the network 140. The HVAC system can provide environmental data such as the current room temperature and humidity and receive commands such as to cool the room to a certain temperature and/or increase a fan speed as an anxiety-reducing countermeasure. For example, the room temperature can be based on a thermometer or other sensor which measures the temperature, or based on a setting such as a temperature set point of the system.

A motion sensor 126 of the treatment room of the medical provider may also be in communication with the network 140. The motion sensor can provide data such as to indicate when an additional person enters the room where the patient is located. This type of data can be considered to be environmental data. The mere presence of an additional person such as a medical provider can be anxiety-inducing. Moreover, a change in the patient's health data such as an increase in heart rate that occurs immediately after detecting the presence of an additional person is a strong indication that the patient's anxiety level has increased.

Multiple types of data can be combined to make a reliable determination that a patient is anxious, while a single type of data provides a less reliable determination that a patient is anxious. Different types of data can be weighted differently according to how strongly they are correlated with inducing anxiety.

In one approach, the rate at which the data is evaluated to determine the status of the patient can vary. For example, the data can be evaluated at a low rate, e.g., once every 30 second, until a trigger event occurs that indicates a threshold level of anxiety is present, e.g., 5 out of 10, after which the data can be evaluated at a higher rate, e.g., once every 10 seconds, to more quickly capture changes/increases in the anxiety level. Additionally, a low number of different types of data, e.g., one type such as heart rate, can be evaluated until a trigger event occurs that indicates a threshold level of anxiety is present, e.g., 5 out of 10, after which one or more additional types of data, e.g., respiratory rate and body temperature, can be evaluated, to more quickly and accurately capture change/increases in the anxiety level.

A presence detector 127 of the treatment room of the medical provider may also be in communication with the network 140. The presence detector can provide data such as the identification of a person who enters the room where the patient is located. This type of data can be considered to be environmental data. The presence of a particular person, e.g., a particular medical provider can be anxiety-inducing. For example, the presence of a provider's assistant such as a doctor's assistant may correlate to a low increase in anxiety while the presence of a doctor may correlate to a higher increase in anxiety. The presence detector 127 may identify the person entering the room such as from a Bluetooth® signal from a smart watch, smart phone or other device worn or carried by the provider. Moreover, an image recognition process could be performed to identify the person entering the room or otherwise moving closer to the patient.

The patient health data, equipment data and environmental data, can be provided via a network 140 to a server 150 which implements one or more models 165 such as including a LLM. In one approach, the network 140 includes a router 175 at the premises of the medical office that communicates wirelessly with the patient health monitoring devices 110, the equipment 130 and the AC system 125. The router can communicate with off-premises equipment including the server 150.

The model can correlate the data it receives to an anxiety level that is then communicated back to computing devices of the patient and/or medical provider. For example, a laptop or PC 160, which includes practice management or other software, smart watch 180, or other provider UI 170 such as a tablet or phone can be used to display information regarding the patient's anxiety level or other status.

It is also possible to provide measurement data (vital signs, environment, etc.) with the correlated anxiety level or by itself. One approach is to provide both the anxiety level and corresponding raw information that was used to determine the anxiety level.

A scent injector 128 can be provided to inject a calming scent into the treatment room. In one approach, the scent injector works by attaching a diffuser unit to the office's HVAC system 125 to distribute a fragrance. A diffuser unit holds a scent oil which is atomized into a fine mist using cold-air diffusion, for example. This mist is released into the HVAC ductwork, where a fan circulates it throughout the office. In another approach, the scent injector operates independently of the HVAC system to provide a more localized effect, such as on or associated with a dental chair or other equipment.

An audio system 129 can be used to play music, sound effects such as nature sounds, or white noise to block out or muffle background noise.

A lighting system 131 can be used to control lights in the treatment room such as ambient lighting from ceiling fixtures, for example. Other lights may be part of the dental operatory equipment 130, such as an operatory or dental light. Other systems 132 can be provided such as to control automated blinds on windows.

The systems 125-129, 131, and 132 may be considered to provide environmental data or otherwise control an environment of the treatment room.

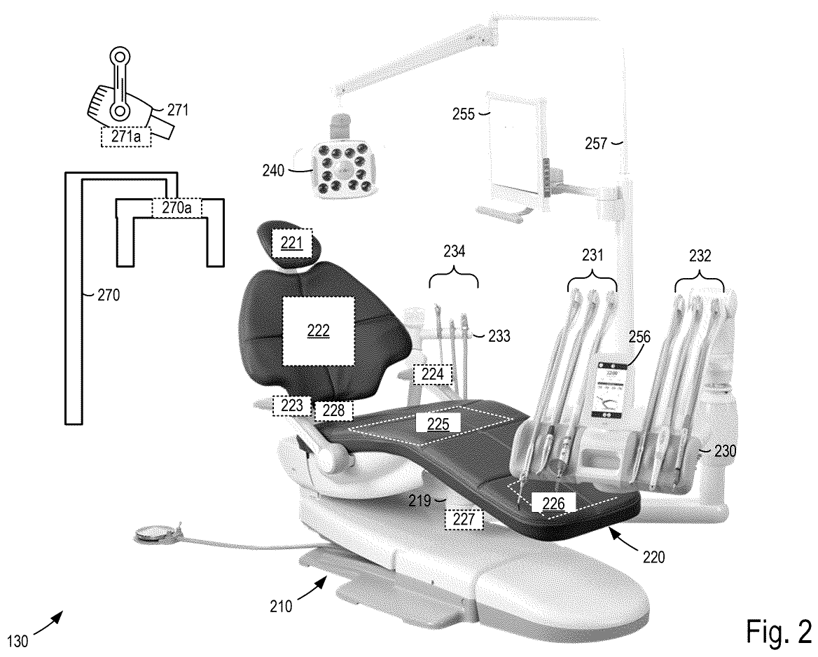

FIG. 2 depicts an example of the dental operatory/treatment room equipment 130 of FIG. 1, in accordance with various embodiments. The treatment equipment includes a chair apparatus 210 including a patient chair 220 mounted on a platform 219, and dental equipment such as a console 230 with tools 231 and 232, a support arm 233 with tools 234, and a light 240. For example, tools such as a drill, suction device and an air-water syringe are commonly used.

A first example UI device 255 is mounted to an arm 257 and may be visible to the patient and/or provider. A second UI device 256 is mounted to the console 230 and may be visible to the provider. The UI devices may have touchscreens to receive user commands.

The chair apparatus may include various pressure sensors, e.g., equipment sensors, that detect movement and/or a position of the patient. Each example sensor represents one or more sensors. For example, a head sensor 221 can detect when the patient leans their head back into the chair and obtain data such as a pressure applied and a duration of the applied pressure. A relatively high pressure, duration and/or repeated movement of the head may correlate with an anxious state. Movement of the head to one side could be detected as a sign of anxiety such as when the patient looks away when a tool or light is directed at them by the provider.

An upper body sensor 222 can detect, e.g., when the user shifts their weight in the chair from side to side or pushes back into the chair. A relatively high amount of shifting and/or pushing may correlate with an anxious state. A camera could also be used to monitor when the user shifts their weight or otherwise changes their body position.

A lower body sensor 225 can also detect, e.g., when the user shifts their weight in the chair from side to side or pushes back into the chair.

Armrest sensors 223 and 224 can detect, e.g., a pressure with which the user is gripping the armrests. A relatively high gripping pressure and/or duration may correlate with an anxious state.

A leg sensor 226 can also detect, e.g., an amount of leg movement by the user. A relatively high amount of movement may correlate with an anxious state.

Although a footrest is not depicted here, if a footrest is provided, sensors may also be included to determine, e.g., if the user is pushing on it with their feet. A relatively large pushing pressure and/or duration may correlate with an anxious state.

A sensor 227 on the platform 219 can also detect an amount of movement of the user. A relatively large amount and/or duration of movement may correlate with an anxious state.

A sensor 228 can be used to detect the angle of recline of the chair. A larger recline may be associated with a higher level of anxiety since most dental procedures are performed while the patient is reclining.

The treatment room can include other equipment such as an X-ray system 271 which is used to create 2D images of the teeth, or a Cone Beam Computed Tomography (CBCT) system 270 which is used to create 3D images of the teeth. These imaging systems can be used to scan the patient while the patient remains seated in the chair, in one approach. Sensors 271a and 270a may indicate when the X-ray system or CBCT system, respectively, is in use. The use of these imaging devices may correlate with an increased level of anxiety.

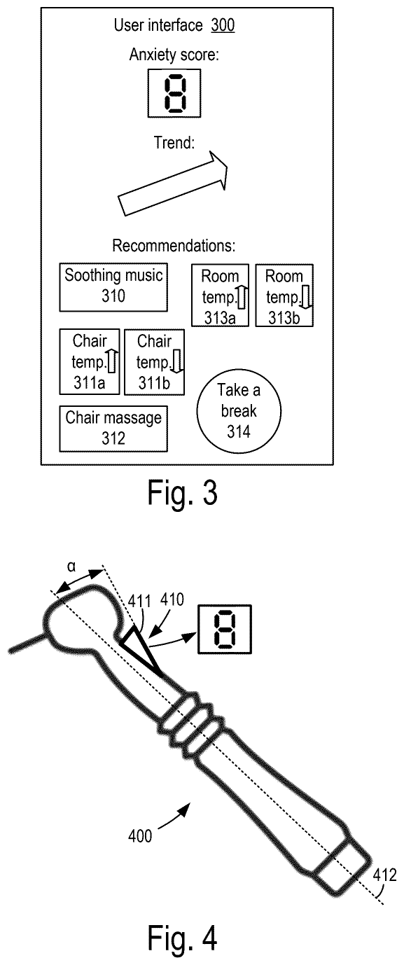

FIG. 3 depicts an example user interface 300 for use in the system of FIG. 1, in accordance with various embodiments. The UI 300 can be provided on the laptop or PC 160, smart watch 180, or other provider or patient UI 170 such as a tablet or phone as in FIG. 1, for example, to display information regarding the patient's anxiety level. The UI could be part of the dental/medical product and can show patient anxiety and/or vital signs. In this example, the anxiety level is expressed as a number or score that can range from a minimum to a maximum, e.g., 1-10. The score in this example is 8, which denotes a relatively high level of anxiety. Another option is to use one or more lights that are color-coded, where green, yellow and red denote low, medium or high anxiety, respectively.

Other options include an audible tone or chime that sounds when the anxiety level exceeds a threshold. Other options include a vibration such as of a piece of equipment used by the provider or on a device such as a smart watch or other wearable worn by the provider. Another example notification could involve a text message indicating the anxiety status sent to a device of the provider. There could also be an indication (e.g., light, audio device, etc.) remotely in the room.

The UI 300 can further indicate a trend of the anxiety level such as by an arrow. In this example, the arrow indicates an increasing trend. The trend can be taken over a relevant period of time such as a few minutes.

The UI can further include touch-selectable icons that allow the medical provider or the patient to start one or more anxiety-reducing countermeasures. The icons can be touched to enter a command. In addition to a touch command, a voice command or physical button press can be used. For example, an icon 310 can start playing soothing music over a speaker in the treatment room or drive a patient entertainment system. An icon 311a or 311b can start a chair warming or cooling process, respectively, such as starting a process to circulate air through holes in the cushions of the chair if it is so equipped and/or to turn on a fan and/or heating element proximate to the chair. An icon 312 can start a chair massage process if the chair is so equipped. An icon 313a or 313b can increase or reduce the room temperature, respectively, such as by operating the HVAC system 125. An anxious patient may tend to feel warm so that reducing the temperature is helpful, for instance. The countermeasures can also include increasing the temperature via the chair and/or the HVAC system 125 if that makes the patient more comfortable.

In one approach, the one or more anxiety-reducing countermeasures can be started automatically when an increased anxiety level is detected.

In one approach, the countermeasures can be phased in as the anxiety level increases. For example, if the anxiety level is above a first threshold, e.g., 5 of 10, a first countermeasure may be started such as playing soothing music. If the anxiety level increases further above a second threshold, e.g., 7 of 10, a second countermeasure may be started such as cooling the chair. If the anxiety level increases further above a third threshold, e.g., 9 of 10, a third countermeasure may be started such as reducing the room temperature. Each additional countermeasure can be added while the previous countermeasures remain in place. The countermeasures can similarly be stopped as the anxiety level decreases below the threshold. A hysteresis may be used so that the threshold to stop or reduce a countermeasure is lower than a threshold to start or increase the countermeasure.

In another example, the severity of the countermeasure is set as an increasing function of the anxiety level. For example, if the anxiety level increases, a countermeasure of cooling the room may be implemented. If the anxiety level increases above a first threshold, the room may cooled to a first temperature, and if the anxiety level increases further above a second threshold, the room may be cooled to a second temperature which is lower than the first temperature. In another example, the countermeasure of cooling the room can involve increasing a fan speed of the HVAC system as the anxiety increases.

It is also possible to associate different levels of stress-reduction effectiveness with different countermeasures, and to select a countermeasure having a stress-reduction effectiveness level corresponding to the anxiety level. For example, when the anxiety level is relatively high, a countermeasure having a relatively high stress-reduction effectiveness level is selected. For instance, a countermeasure of playing soothing music may have a relatively low stress-reduction effectiveness level, and a countermeasure of cooling the room with an HVAC system may have a relatively high stress-reduction effectiveness level. The level of stress-reduction effectiveness of a countermeasure can be determined from testing, research or other approaches.

Other examples of countermeasures including reducing the brightness and/or increasing the warmth of the light 240 (increasing the color temperature) when the anxiety level is high. For example, a warm color such as yellow or orange can be used in place of a cool white. A warm light has a longer wavelength than a cool light. Another example countermeasure is to change the color of light in the room including the ceiling light, cabinetry light, dental light, or loupes.

Another example countermeasure is to inject a scent into the area of the procedure, such as a lavender, chamomile, vanilla, or sandalwood scent, which have calming properties, using a scent injector 128. The rate of injecting the scent can be increased as the anxiety level increases.

Other examples of countermeasures including changing the operation of the tools used in a procedure. For example, a tool may have a quiet mode or a slower operating speed that can be used when the patient's anxiety level is high. The countermeasures could potentially be triggered automatically, without intervention of the provider, based on real-time data of the patient's anxiety level. An example of a countermeasure is a noise cancellation or noise masking technique. Another example of a countermeasure is adjusting a water and/or air flow rate to instruments of the treatment center. Another example of a countermeasure is a message to consider using a different tool. The message can be transmitted to the provider on a UI. For example, a message can indicate that a different tool should be used in place of a drill.

In another option, a warning light 314 may suggest that the provider take a break from the procedure if the patient's anxiety level is very high, e.g., above a threshold level. The UI may further include the option to start and stop tracking of a patient's anxiety level.

In one approach, the server 150 determines the anxiety level and one or more corresponding anxiety-reducing countermeasures and transmits this information to the UI. In another approach, the server transmits the anxiety level back to the UI and the UI determines the one or more corresponding anxiety-reducing countermeasures based on the anxiety level. This allows the treatment site to customize the countermeasures that are used.

In one option, the countermeasures are customized to the patient.

FIG. 4 depicts an example dental tool 400 with a user interface 410 for use in the system of FIG. 1, in accordance with various embodiments. By integrating the UI into a tool, the provider can more easily see the anxiety level even when engrossed in a procedure that involves using the tool while focusing on the patient's mouth, for instance. In an example implementation, the UI is integrated into a dental drill as this may be the most anxiety-inducing tool among the dental tools. The UI 410 extends up from the side of the tool in this example for increased visibility to the user, but could be flush with the surface of the tool in other implementations. The UI has a surface 411 which is at an angle α (e.g., less than 90 degrees) relative to a longitudinal axis 412 of the tool. In another example, the UI is flush with the side of the tool. The presence of the UI on the tool is useful as the provider may not be able to see other UI screens when performing close up work in the patient's mouth, for example.

Another option is to provide the anxiety level on a heads-up display that is in the line of sight of the provider, such as on smart goggles or smart glasses worn by the provider, or on a smart watch.

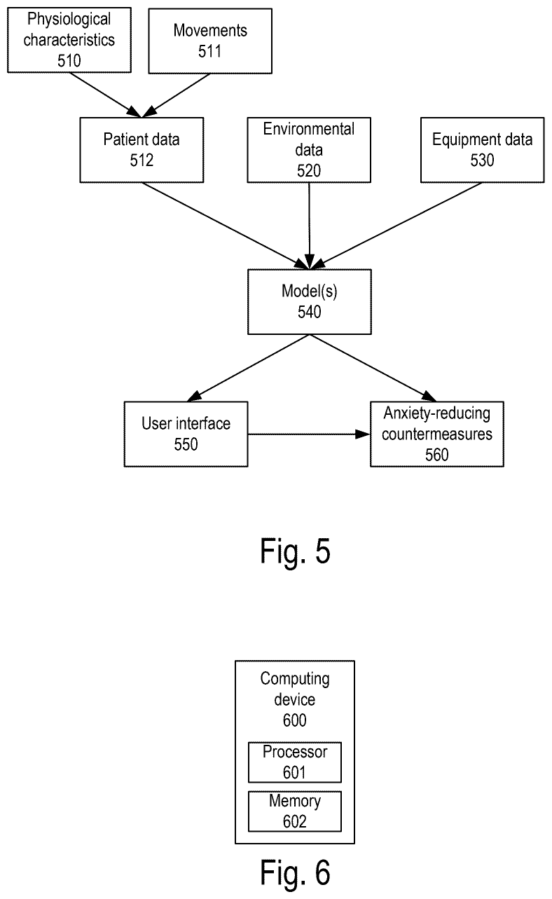

FIG. 5 depicts an example implementation of the system of FIG. 1, in accordance with various embodiments. Patient sensors 512 obtain information such as physiological characteristics, e.g., health data (block 510) and movements (block 511) of a patient. This can include many types of data as discussed including heart rate, blood pressure, respiratory rate, breathing changes, shaking or trembling, muscle tension, body temperature, blood oxygen level (oxygen saturation level), presence or amount of sweating, data regarding sleep disruption, the patient's voice, and images of the patient's eyes and face.

Environmental data 520 can include data such as the temperature and humidity of the room. For example, a higher temperature and humidity may correlate with a less comfortable environment that is anxiety-inducing. The environmental data could also include audio from one or more microphones to detect an ambient noise level. The microphones to detect an ambient noise level can be the same as, or different than, the microphone 152 used to record the patient's voice. A higher noise level may correlate with a less comfortable environment that is anxiety-inducing.

In another option, the microphone can detect a type of tool that the provider is using. For example, the high pitch of a dental drill may be recognizable. The use of such a tool may correlate with a higher anxiety. Other examples of anxiety-inducing powered tools include a polisher, which is another air-driven hand-held device that creates noise and vibration, and a suction device, which removes excess saliva and water from the mouth, but can be anxiety-inducing due to the noise of the vacuum and the sensation of a tube in the mouth which can cause gagging.

The microphones can also identify a procedure being performed. The audio detected by the microphone can be compared to a library of sounds at the model 165 to try to detect a matching sound, such as to identify a particular powered tool being used among multiple types of powered tools, and to set or adjust an anxiety level or other status of the patient accordingly. For example, an anxiety level on a scale of 1-10 can be increased by 5 points if the dental drill is detected, 3 points if the polisher is detected, and 2 points if the suction device is detected.

Matching a detected power tool sound to a library of sounds can involve digital signal processing and machine learning techniques. The process can include audio feature extraction from the detected sound and the library, followed by a comparison or classification step.

Initially, a library of sounds may be created. The library can contain a variety of annotated audio files for each power tool to be identified, recorded in different conditions (e.g., different environments, usage styles, and background noise levels). The files may be annotated to identify the type of tool.

Instead of comparing raw audio waveforms, which is inefficient, key features that characterize the sound can be extracted. Example audio features include: Mel-Frequency Cepstral Coefficients (MFCCs), spectral features, and temporal features. MFCCs are computationally lightweight, making them suitable for real-time applications. Spectral features describe the frequency content of the sound, such as spectral centroid, spectral flux, and sub-band energy ratios. Temporal features describe how the sound changes over time, including short-time energy (volume), zero-crossing rate, and envelope characteristics (attack, sustain, decay, release).

A machine learning model can be trained to recognize the patterns in the extracted features. Algorithms such as Support Vector Machines (SVM), K-Nearest Neighbors (KNN), Hidden Markov Models (HMMs), or deep learning models such as Convolutional Neural Networks (CNNs) can be used. CNNs, in particular, can analyze visual representations of the audio data, like spectrograms, to classify sounds effectively. The model is trained using the labeled feature data from the library. This teaches the computer to associate specific feature patterns with particular power tools.

Once the model is trained, it can be put into use. When a power tool sound is detected, the incoming audio stream is processed to reduce background noise and isolate the target sound. The same feature extraction methods used for the library are applied to the new sound. The extracted features of the new sound are fed into the trained machine learning model. The model then outputs a classification result, identifying which power tool from the library the sound most closely matches. This result is then used to set or adjust an anxiety level of the patient, as mentioned.

Equipment data 530 can also be provided. One example is the intensity of the dental operatory lamp, where a higher intensity is more anxiety-inducing. Another example is the color of the lamp, where a cooler color is more anxiety-inducing. Another example is the type of tool being used in a procedure. For example, a console that holds the tool may have a sensor that is triggered when a specific tool is being used. The type of tool being used may correlate with a likelihood of inducing anxiety, as discussed. The dental system can know what equipment is being operated based on the activation of circuits within the system. For example, a controller within the equipment can know when a lamp is turned on, and the setting of the lamp. The controller can also know when a particular tool is activated when power is supplied to that tool. In one approach, software at the controller keeps track of the equipment that is currently in use and the associated settings.

The patient, environmental, and equipment data is provided to one or more models 540 such as including a LLM. The model determines correlations between the data and an anxiety level based on its training, and transmits data indicating the anxiety level to a user interface 550 such as discussed in connection with FIGS. 3 and 4. In one approach, as discussed, the UI can be used by the provider or patient to start one or more anxiety-reducing countermeasures (block 560), or the output of the model can be used directly by the block 560 to automatically start the one or more anxiety-reducing countermeasures.

FIG. 6 depicts an example computing device for use in the system of FIG. 1, in accordance with various embodiments. The computing device 600 can represent any of the computing devices discussed herein such as a user-worn device, dental or other medical equipment, a server, a laptop, or a PC. The computing device 600 can represent a device on which a UI is provided. The computing device 600 includes a memory 602 to store instructions and a processor 601 to execute the instructions to provide the features described herein. The memory 602 may comprise a non-transitory, tangible storage medium. The computing device 600 can be on-site, at the treatment room or otherwise in the provider's facility, or remote from the treatment room and facility.

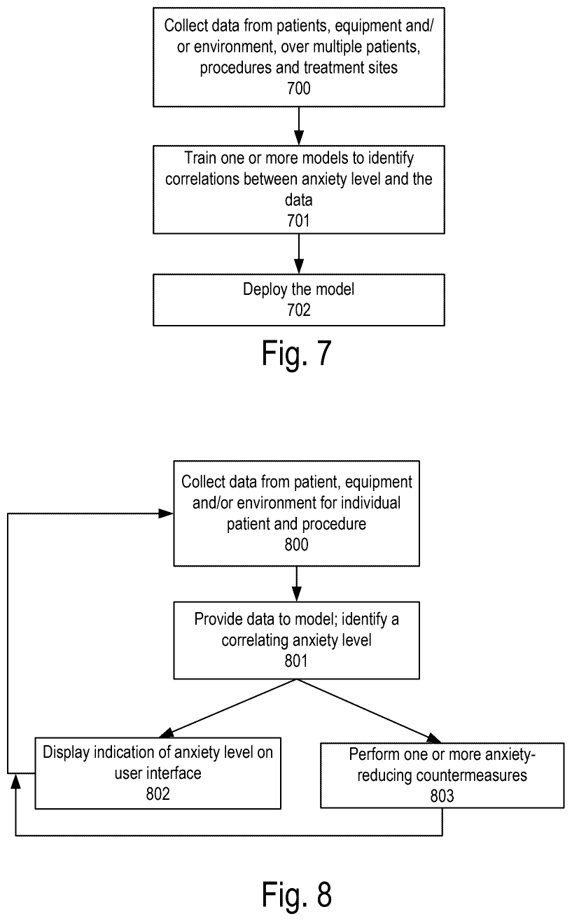

FIG. 7 depicts an example process to train a model to correlate the status of a population of patients to health data, equipment data and environmental data, in accordance with various embodiments. Step 700 includes collecting data from patients, equipment, and/or the environment (e.g., of the treatment room) over multiple patients and procedures and treatment sites. Step 701 includes training one or more models to identify correlations between anxiety level and the data. The training can be made for different procedures together, or can be specific to a procedure, such as cleanings, fillings, extractions, crowns, root canals, and oral exams at a dental office. The type of procedure can be determined in different ways. In one approach, equipment data such as on a console, as mentioned, indicate that certain tools are used that correlate to a certain procedure. For example, use of a drill correlates to providing a filling in a tooth. The sequence and duration of use of the tools can correlate to a certain procedure.

The training can also be separate for different population groups such as by age and gender. Different models can be used for different groups, procedures and/or treatment sites. Regarding treatment sites, the training can be separate for the patients of a given medical office or group of affiliated offices, for instance.

Step 702 includes deploying the model. In one approach, software for the model is run at a central server, in a client-server model, so that it can be maintained and updated for use by multiple medical provider offices. The server can be in the cloud or on-premises. One or more computing devices of the medical provider and/or patient or computing devices otherwise at the treatment site will have client-side software installed while the remote server has server-side software installed.

In another approach, the software for the model is run locally at one or more computing devices of the medical provider and/or patient without communicating with a remote server. For example, the laptop or PC 160 may acts as a server and run the server-side software while one or more computing devices of the medical provider and/or patient or otherwise at the treatment site run the client-side software. A dental device or UI (e.g., smartwatch/phone) can be used as well.

FIG. 8 depicts an example process to use a model to determine the status of an individual patient based on health data, equipment data and environmental data, in accordance with various embodiments. Step 800 includes collecting data from patients, equipment, and/or the environment for an individual patient and procedure. Step 801 includes providing the data to a model and identifying a correlating/corresponding anxiety level. Step 802 includes displaying an indication of the anxiety level on a user interface, and step 803 includes performing one or more anxiety-reducing countermeasure.

The process can be repeated periodically during a time period of interest, e.g., during a patient's visit, including during a procedure and potentially before and/or after the procedure as well.

In one option, a patient profile is developed for an individual patient to note information such as their anxiety level from previous office visits and procedures. Anxiety-reducing countermeasures can be taken proactively, e.g., before the patient becomes very anxious during a procedure, such as by setting the lights, temperature and music when the patient first arrives in the office. The provider can also decide to use different equipment or otherwise modify a procedure for a patient that is known to become anxious in general or anxious during a particular procedure. The provider can decide to allocate more time for an anxious patient. The patient profile can also indicate that a patient is more or less anxious with different providers and decide to schedule the patient with a provider who is less anxiety-inducing. The provider can be informed by the model that certain anxiety-reducing countermeasures should be used.

The anxiety score of an individual patient can be adjusted or coded base on their profile. For example, if a patient has had a relatively low anxiety level such as 2 out of 10 in previous visits and has a score of 6 out of 10 in a current visit, this can be coded as an urgency of red or high anxiety since the score has increased by more than a threshold amount, e.g., 3. The coding or urgency of the anxiety is therefore based on an increase in the anxiety level relative to one or more past visits including an average or mean of past scores.

If a patient has had a moderately high anxiety level such as 5 out of 10 in previous visits and also has a score of 6 out of 10 in a current visit, this can be coded as an urgency of yellow or medium anxiety since the score has not increased by the threshold amount, e.g., 3.

The coding of the urgency of the anxiety level can therefore be customized to the individual patient.

In sum, the solutions herein provide a number of features. The solutions integrate health-monitoring technology from devices, sensors, and data such as a fitness/tracker or smart watch (one example supplier is the Apple Watch®), equipment in a treatment room or operatory such as a dental chair (one example supplier is A-dec, Newberg, Oregon), dental or other medical equipment and delivery systems, third-party sensors and vital signs devices, medical/dental practice management software, and drives them in a Large Language Model that uses the inputs to provide recommendations around the patient's vital signs and possible anxiety.

The output, including recommendations and data inputs, may be presented to the provider or patient on their wearable or portable device, on a dental user interface or other method of indication, and/or a computer system which may include practice management or other dental software solution systems designed for use in dental or medical practice environments. This system collects real-time physiological data that may indicate a patient's level of anxiety, including heart rate, respiratory rate, blood pressure, and other factors like muscle tension, shaking or trembling, breathing changes, and potential sleep disruption.

These inputs may be combined with product or operatory sensors and data. Operatory sensors could include a plurality of sensors located within the dental ecosystem such as accelerometers to measure vibration or inclination, microphones to measure noise frequency and amplitude, instrument air, water, speed, torque, and directional information, light sensors to measure the light in the environment or near the patient, temperature and humidity sensors to measure the environment, pressure sensors, occupancy sensing of the patient or staff, motion sensors to understand movement, weight and pressure sensors, and more. These may be combined with dental clinic data or activities such as chair positioning and movement, delivery system instrument use, movement, feedback, clinical workflow, practice management information, information from third party or other dental software solutions, dental light settings, and more.

The data usage and insights from the treatment center, furniture, portables, practice management, third party software solutions such as X-Ray or Cone Beam Computed Tomography (CBCT), or other software or data in the dental clinic. The data is securely transmitted to an on-premises cloud platform and processed through an AI-driven health analytics model that leverages machine learning (LLM). This model analyzes vital signs and other inputs to assess the patient's stress and anxiety levels.

The processed insights can then displayed on the dental user interface, staff wearable, staff portable device, practice management interface, other software in the clinic, patient wearable or device, and/or other method of conveying the information involving sounds, readouts, lights, etc. providing practitioners with real-time and historical feedback and recommended adjustments or interventions to ensure patient comfort and improve the care experience. Future enhancements include tracking additional metrics such as pupil dilation and sweating.

This system is structured to seamlessly connect the inputs and outputs noted above through robust connections, Application Programming Interface (API) integrations and machine learning models. It can be targeted at dental and medical practices aiming to enhance the patient experience, better manage patient anxiety, and improve the quality of care through data-driven insights.

The solutions include the seamless data integration and transactions between systems noted above. For example, if vital signs or anxiety recommendations are formed and presented, these records can be saved in the practice management software.

The solutions solve a problem not previously addressed: monitoring and insights around patient anxiety. Further, the solutions automate the data integrations, display, and data transactions so that no manual recording is required for entering the data. Providing awareness and insights of patient vital signs will also improve patient care during procedures and allow the medical provider to provide awareness to the patients if there is a recommendation for them to consult their primary physician.

Some non-limiting examples of various embodiments are presented below.
Example 1 includes a system, comprising: one or more computing devices configured to receive health data of a patient in a treatment room, receive equipment data of equipment in the treatment room, and process the health data and the equipment data based on a model to determine a status of the patient; and a user interface device in communication with the one or more computing devices and configured to provide an output based on the status of the patient.
Example 2 includes the system of Example 1, wherein the health data comprises at least one of heart rate, blood pressure, respiratory rate, breathing changes, shaking or trembling, muscle tension, body temperature, blood oxygen saturation, presence or amount of sweating, or sleep disruption.
Example 3 includes the system of Example 1 or 2, wherein the equipment data indicates at least one of movement of the patient in a chair of the treatment room, a pressure applied to an armrest of the chair by the patient, a height of the chair, or an angle of recline of the chair.
Example 4 includes the system of any one of Examples 1-3, wherein the equipment data indicates at least one of a brightness or a color setting of an operatory light, user light or overhead room light of the treatment room.
Example 5 includes the system of any one of Examples 1-4, wherein the equipment data indicates at least one of a type of tool being used or a speed of a tool being used.
Example 6 includes the system of any one of Examples 1-5, wherein the health data comprises image data of one or both eyes of the patient, and the one or more computing devices are configured to identify at least one of movement of the one or both eyes of the patient, or a dilation of the one or both eyes of the patient based on the image data of the one or both eyes to determine the status.
Example 7 includes the system of any one of Examples 1-6, wherein the health data comprises image data of a face of the patient, and the one or more computing devices are configured to identify a facial expression of the patient based on the image data of the face to determine the status.
Example 8 includes the system of any one of Examples 1-7, wherein the health data comprises image data of the patient's body, and the one or more computing devices are configured to identify a body position of the patient based on the image data of the patient's body to determine the status.
Example 9 includes the system of any one of Examples 1-8, wherein the health data comprises audio of a voice of the patient, and the one or more computing devices are configured to detect at least one of a change or a quality of the voice of the patient to determine the status.
Example 10 includes the system of any one of Examples 1-9, wherein the one or more computing devices are configured to receive environmental data of the treatment room, and to process the health data, the equipment data and the environmental data with the model to determine the status of the patient.
Example 11 includes the system of Example 10, wherein the environmental data indicates that one or more additional persons have entered the treatment room and an identification of the one or more additional persons.
Example 12 includes the system of Example 10 or 11, wherein the environmental data indicates at least one of a temperature, a humidity level or an ambient noise level of the treatment room.
Example 13 includes the system of any one of Examples 1-12, wherein the model comprises a large language model (LLM) that is trained with data from a patient population.
Example 14 includes the system of any one of Examples 1-13, wherein the status of the patient comprises an anxiety level.
Example 15 includes the system of Example 14, wherein based on the anxiety level, the user interface device is configured to provide one or more selectable options for anxiety-reducing countermeasures and/or the one or more computing devices are configured to automatically start one or more anxiety-reducing countermeasures.
Example 16 includes the system of Example 14 or 15, wherein the one or more computing devices are configured to code an urgency of the anxiety level based on an anxiety level of the patient during one or more previous procedures.
Example 17 includes the system of any one of Examples 1-16, wherein: the one or more computing devices are configured to detect a sound of a powered tool in the treatment room, compare the sound to a library of sounds to identify the powered tool, and determine the status of the patient based on the identified powered tool.
Example 18 includes the system of any one of Examples 1-17, wherein: based on the status of the patient, the one or more computing devices are configured to start one or more anxiety-reducing countermeasures, automatically and/or based on a command made via the user interface; and the one or more anxiety-reducing countermeasures comprise at least one of adjusting a temperature of the treatment room, adjusting a temperature of a patient chair of the treatment room, playing soothing music, activating a patient entertainment system, activating noise cancellation techniques, controlling patient media, injecting a scent, reducing a brightness of a light, or increasing a color temperature of a light.
Example 19 includes the system of any one of Examples 1-18, wherein the one or more computing devices are configured to receive the health data from at least one of a wearable device of the patient or the equipment in the treatment room.
Example 20 incudes an apparatus, comprising: a memory to store instructions; and a processor configured to execute the instructions to: receive health data of a patient and equipment data of a treatment room of the patient; determine a correlation of an anxiety level of the patient to the health data and the equipment data; and transmit data indicating the anxiety level to a user interface device.
Example 21 includes the apparatus of Example 20, wherein the processor is configured to execute the instructions to transmit data indicating one or more anxiety-reducing countermeasures to the user interface device based on the anxiety level.
Example 22 includes the apparatus of Example 20 or 21, wherein the processor is configured to execute the instructions to transmit data indicating an urgency of the anxiety level to the user interface device.
Example 23 includes the apparatus of any one of Examples 20-22, wherein the processor is configured to execute the instructions to receive environmental data of the treatment room, and to determine a correlation of the anxiety level to the environmental data.
Example 24 includes a computer-implemented method, comprising: receiving health data of a patient and equipment data of a treatment room of a patient; determining a correlation of an anxiety level of the patient to the health data and the equipment data; and transmit data indicating the anxiety level to a user interface device.
Example 25 includes the computer-implemented method of Example 24, further comprising transmitting data indicating one or more anxiety-reducing countermeasures to the user interface device based on the anxiety level.
Example 26 includes the computer-implemented method of claim 24 or 25, further comprising transmitting data indicating an urgency of the anxiety level to the user interface device.
Example 27 includes an apparatus comprising means to perform the computer-implemented method of claim 24 or 25.
Example 28 includes a non-transitory, computer-readable medium comprising instructions which, when executed by a processor, cause the processor to: receive health data of a patient and equipment data of a treatment room of the patient; determine a correlation of an anxiety level of the patient to the health data and the equipment data; and transmit data indicating the anxiety level to a user interface device.
Example 29 includes the non-transitory, computer-readable medium of Example 28, wherein the determining of the correlation uses a large language model (LLM) that is trained with data from a patient population.
Example 30 includes the non-transitory, computer-readable medium of Example 28 or 29, wherein the instructions, when executed by a processor, cause the processor to transmit data indicating one or more anxiety-reducing countermeasures to the user interface device based on the anxiety level.
Example 31 includes the non-transitory, computer-readable medium of any one of Examples 28-30, wherein the instructions, when executed by a processor, cause the processor to transmit data indicating an urgency of the anxiety level to the user interface device.

Although certain embodiments have been illustrated and described herein, it will be appreciated by those of ordinary skill in the art that a wide variety of alternate and/or equivalent embodiments or implementations calculated to achieve the same purposes may be substituted for the embodiments shown and described without departing from the scope. Those with skill in the art will readily appreciate that embodiments may be implemented in a very wide variety of ways. This application is intended to cover any adaptations or variations of the embodiments discussed herein. Therefore, it is manifestly intended that embodiments be limited only by the claims and the equivalents thereof.

What is claimed is:

1. A system, comprising:
one or more computing devices configured to receive health data of a patient in a treatment room, receive equipment data of equipment in the treatment room, and process the health data and the equipment data based on a model to determine a status of the patient; and
a user interface device in communication with the one or more computing devices and configured to provide an output based on the status of the patient.

2. The system of claim 1, wherein the health data comprises at least one of heart rate, blood pressure, respiratory rate, breathing changes, shaking or trembling, muscle tension, body temperature, blood oxygen saturation, presence or amount of sweating, or sleep disruption.

3. The system of claim 1, wherein the equipment data indicates at least one of movement of the patient in a chair of the treatment room, a pressure applied to an armrest of the chair by the patient, a height of the chair, or an angle of recline of the chair.

4. The system of claim 1, wherein the equipment data indicates at least one of a brightness or a color setting of a light of the treatment room.

5. The system of claim 1, wherein the equipment data indicates at least one of a type of tool being used or a speed of a tool being used.

6. The system of claim 1, wherein the health data comprises image data of one or both eyes of the patient, and the one or more computing devices are configured to identify at least one of movement of the one or both eyes of the patient, or a dilation of the one or both eyes of the patient based on the image data of the one or both eyes to determine the status.

7. The system of claim 1, wherein the health data comprises image data of a face of the patient, and the one or more computing devices are configured to identify a facial expression of the patient based on the image data of the face to determine the status.

8. The system of claim 1, wherein the health data comprises image data of the patient's body, and the one or more computing devices are configured to identify a body position of the patient based on the image data of the patient's body to determine the status.

9. The system of claim 1, wherein the health data comprises audio of a voice of the patient, and the one or more computing devices are configured to detect at least one of a change or a quality of the voice of the patient to determine the status.

10. The system of claim 1, wherein the one or more computing devices are configured to receive environmental data of the treatment room, and to process the health data, the equipment data, and the environmental data with the model to determine the status of the patient.

11. The system of claim 10, wherein the environmental data indicates that one or more additional persons have entered the treatment room and an identification of the one or more additional persons.

12. The system of claim 10, wherein the environmental data indicates at least one of a temperature, a humidity level, or an ambient noise level of the treatment room.

13. The system of claim 1, wherein the model comprises a large language model (LLM) that is trained with data from a patient population.

14. The system of claim 1, wherein the status of the patient comprises an anxiety level.

15. The system of claim 14, wherein based on the anxiety level, the user interface device is configured to provide one or more selectable options for anxiety-reducing countermeasures and/or the one or more computing devices are configured to automatically start one or more anxiety-reducing countermeasures.

16. The system of claim 14, wherein the one or more computing devices are configured to code an urgency of the anxiety level based on an anxiety level of the patient during one or more previous procedures.

17. The system of claim 1, wherein:
the one or more computing devices are configured to detect a sound of a powered tool in the treatment room, compare the sound to a library of sounds to identify the powered tool, and determine the status of the patient based on the identified powered tool.

18. The system of claim 1, wherein:
based on the status of the patient, the one or more computing devices are configured to start one or more anxiety-reducing countermeasures, automatically and/or based on a command made via the user interface; and
the one or more anxiety-reducing countermeasures comprise at least one of adjusting a temperature of the treatment room, adjusting a temperature of a patient chair of the treatment room, playing soothing music, activating a patient entertainment system, activating noise cancellation techniques, controlling patient media, injecting a scent, reducing a brightness of a light, or increasing a color temperature of a light.

19. The system of claim 1, wherein the one or more computing devices are configured to receive the health data from at least one of a wearable device of the patient or the equipment in the treatment room.

20. An apparatus, comprising:
a memory to store instructions; and
a processor configured to execute the instructions to:
receive health data of a patient and equipment data of a treatment room of the patient;
determine a correlation of an anxiety level of the patient to the health data and the equipment data; and
transmit data indicating the anxiety level to a user interface device.

21. The apparatus of claim 20, wherein the processor is configured to execute the instructions to transmit data indicating one or more anxiety-reducing countermeasures to the user interface device based on the anxiety level.

22. The apparatus of claim 20, wherein the processor is configured to execute the instructions to transmit data indicating an urgency of the anxiety level to the user interface device.

23. The apparatus of claim 20, wherein the processor is configured to execute the instructions to receive environmental data of the treatment room, and to determine a correlation of the anxiety level to the environmental data.
